(19) United States
(12) Patent Application Publication
(10) Pub. No.: US 2009/0306463 A1
(43) Pub. Date: Dec. 10, 2009

(54) Gastric Ring

(76) Inventors: Didier Philippe Mouton, Saint-Priest-En-Jarez (FR); Maxime Sodji, Limoges (FR); Felipe Dela Cruz Vigo, Madrid (ES); Jose Luiz De La Cruz, Leon (ES)

Correspondence Address: DOWELL & DOWELL P.C., 103 Oronoco St., Suite 220, Alexandria, VA, 22314, US

(21) Appl. No.: 12/087,595

(22) PCT Filed: Jan. 15, 2007
(86) PCT No.: PCT/FR2007/000066

§ 371 Date: Oct. 23, 2008

(30) Foreign Application Priority Data
Jan. 16, 2006 (FR) 06 00375

(51) Int. Cl. A61F 2/04

(52) U.S. Cl. 600/37; 606/157

(57) ABSTRACT

The gastric ring has two ends (11, 12) and, between these ends, an intermediate portion (13) of solid section for coming to bear simply against a stomach portion that is to be surrounded. The first end (11) forms an opening (115) in which the second end (12) can be inserted. The second end (12) is suitable for being prevented from moving in said opening (115) in at least two distinct positions, thereby enabling the inside diameter (d′₁) of the ring to be adjusted to its implantation conditions.

The invention relates to a gastric ring of the kind used for gastroplasty operations. Such a ring is designed to surround a portion of the stomach in order to provide a calibration or constriction effect in the context of treatment for obesity.

By way of example, FR-A-2 799 118 discloses the use of a gastric ring provided with an expandable pouch that is filled with physiological liquid as a function of the diameter desired for the gastric constriction that is obtained by means of such a ring. It is relatively complex to control inflation of the pouch and it is necessary to make use of a subcutaneous box, with the associated risk of contamination or leakage.

WO-A-02/096326 enables those drawbacks to be mitigated by providing a gastric ring that does not have an expandable portion and that is designed, in a main portion between its ends, to bear against the gastric wall. Such a ring is entirely effective, however its inside diameter in the closed configuration cannot be adjusted. Unfortunately, depending on the morphology of the patient, on different gastric wall thicknesses, on the patient's eating habits, and on the patient's psychological profile, it can be necessary to make use of different calibration or constriction diameters, so prior art rings need to be provided in several sizes.

The invention seeks more particularly to remedy those drawbacks by proposing a gastric ring that does not require an inflatable portion to be controlled, and that presents an inside diameter that can be modified while it is being put into place in order to match it to different implantation configurations.

To this end, the invention relates to a gastric ring having no expandable portion, the ring comprising two ends with, between those ends, an intermediate portion of solid section that is to come to bear simply against a stomach portion that is be surrounded, a first end of said ring forming an opening in which it is possible to insert the second end. The ring is characterized in that the second end is suitable for being held stationary in the above-mentioned opening in at least two distinct positions.

By means of the invention, the inside diameter of the gastric ring in its utilization configuration can vary as a function of the position in which its second end is prevented from moving in the opening of its first end. This makes it possible to adjust the inside diameter of the gastric ring quickly and reliably.

According to aspects of the invention that are advantageous but not essential, such a ring may include one or more of the following characteristics:
the ring includes portions in relief for blocking the second end in the opening in each of the above-mentioned positions, and in addition to said portions in relief, the ring includes locking means for locking the second end in the opening in each of the above-mentioned positions;
the locking means comprise at least a first orifice formed in one of the ends and at least two second orifices formed in the other end, one of the second orifices coming into alignment with the first orifice in each of the above-mentioned positions, these orifices then forming together a channel through which a member can be passed for holding the ends of the ring relative to each other;
the opening in the second end is provided with a blocking portion in relief that is suitable for co-operating, as a function of the diameter selected for the ring, with one or more complementary blocking portions in relief that are formed on the second end;
the portion in relief for blocking the opening is a tooth that projects from an edge of said opening towards the opposite edge;
the complementary portions in relief formed on the second end are formed by teeth disposed one behind another along a longitudinal axis of the second end;
the teeth in the opening and on the second end have respective surfaces that are inclined relative to a middle axis of the opening and relative to the longitudinal axis of the second end, in a direction that is compatible with moving the second end through the opening so as to tighten the ring;
the teeth have respective surfaces perpendicular to a middle axis of the opening and to the longitudinal axis of the second end, these faces bearing one against another when the second end is prevented from moving in the opening in the first end, in one of the above-mentioned positions;
a volume defined between two adjacent teeth formed on the second end is substantially complementary in shape to the tooth that projects from an edge of the opening;
the intermediate portion of the ring is provided, on each of its longitudinal edges, with an orifice for passing a ligating suture, these orifices being substantially diametrically opposite when the second end is prevented from moving in the opening in one of the above-mentioned positions.

The invention will be better understood and other advantages thereof appear more clearly in the light of the following description of an embodiment of a gastric ring in accordance with the principle of the invention, given purely by way of example and made with reference to the accompanying drawings, in which:

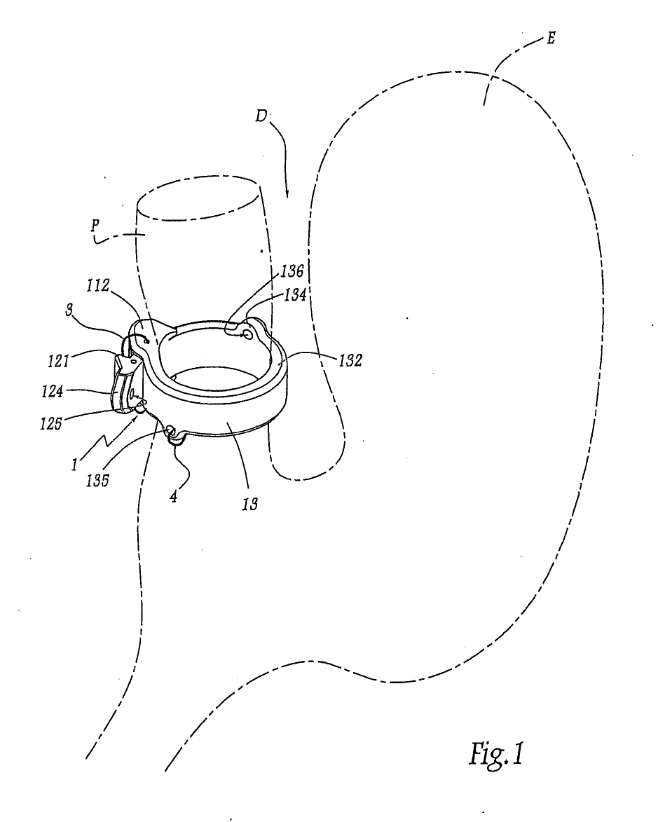

FIG. 1 is a diagrammatic view of a gastric ring in accordance with the invention implanted on a stomach;

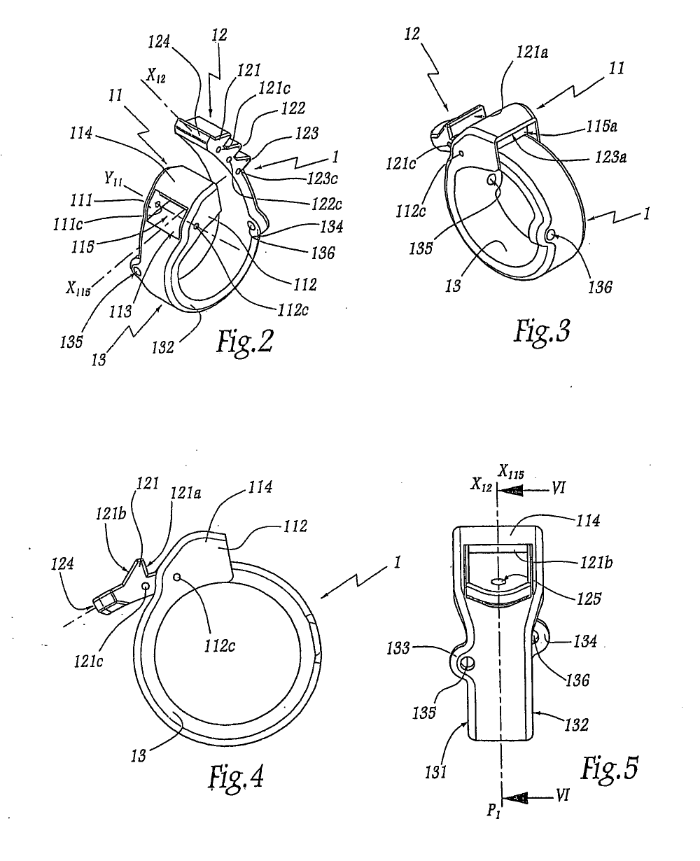

FIG. 2 is a perspective of the FIG. 1 ring in an open configuration;

FIG. 3 is a perspective view of the FIG. 1 ring seen from another angle;

FIG. 4 is a side view of the ring in its configuration of FIGS. 1 and 3;

FIG. 5 is a face view of the ring in its configuration of FIGS. 1, 3, and 4;

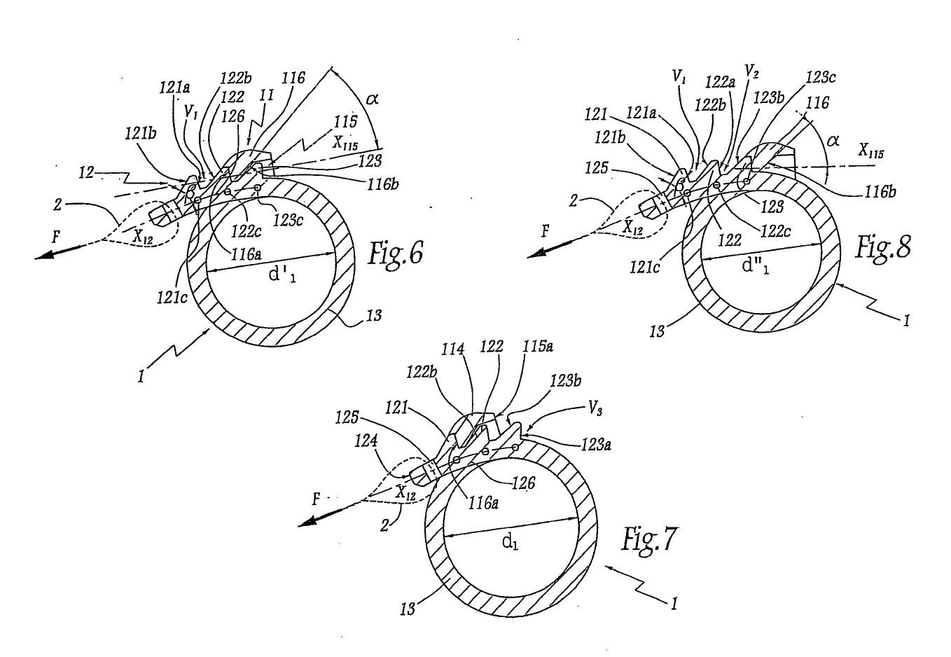

FIG. 6 is a section on line VI-VI of FIG. 5;

FIG. 7 is a section analogous to FIG. 6 when the ring is in another utilization configuration;

FIG. 8 is a section analogous to FIG. 6 when the ring is in yet another utilization configuration; and

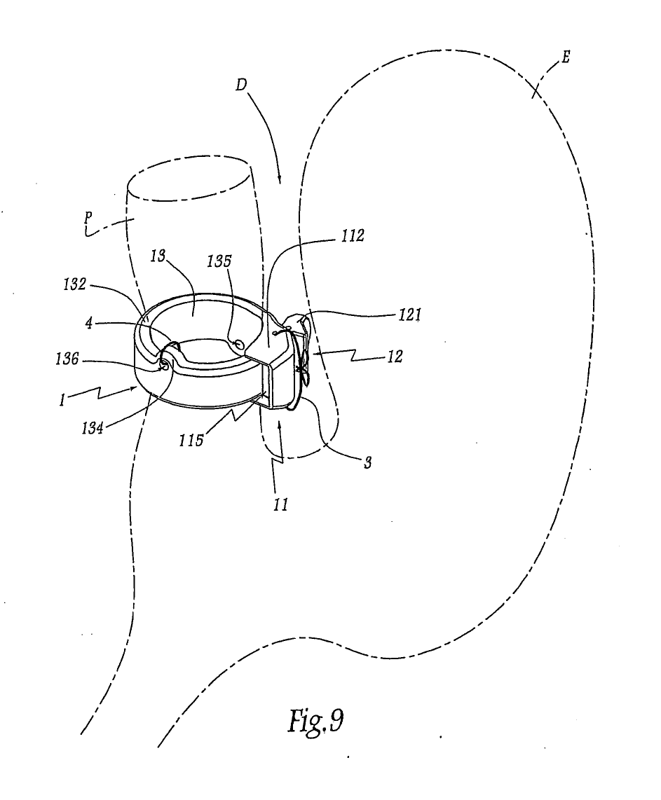

FIG. 9 is a diagrammatic view of another configuration for implanting the ring of the preceding figures.

The ring 1 shown in FIGS. 1 to 9 is a single molded piece of elastomer material. The ring 1 presents a certain amount of elasticity enabling it to be taken from an open position as shown in FIG. 2 to a closed position as shown in FIGS. 1, 3 to 6, and. 9, in which it presents a collar configuration enabling it to surround or squeeze a portion P of the stomach E of a patient, the portion being obtained by creating a cutout D in the stomach.

The ring 1 is generally in the form of a strip having a first end 11 and a second end 12 between which there is defined an intermediate portion 13 of solid section having no expandable portion and designed to bear against and surround, via its inside surface, the portion P.

The end 11 has two partitions 111 that extend from the extension 113 of the portion 13 at the end 11 and that are interconnected by a bar 114. The elements 111 to 114 thus define an opening 115 in the form of a tunnel over the length of the bar 114 as measured parallel to a longitudinal axis X₁₁₅ of the opening 115.

On its inside face facing towards the extension 113, the bar 114 is provided with a tooth 116 that is defined between a surface 116a generally perpendicular to the axis X₁₁₅ and a surface 116b that is inclined relative to said axis by an angle α of about 40°. In practice, the angle α may have a value lying in the range 30° to 60°. The orientation of the surface 116b is such that it approaches the bar 114 by approaching the entry side 115a of the opening 115 that is directed away from the portion 13.

The end 12 extends in line with the portion 13. Reference X₁₂ designates the longitudinal axis that is curved in the plane of FIGS. 6 to 8, given the curved nature of the end 12. The end 12 carries three teeth 121, 122, and 123 that are formed respectively on the outside of the end 12 and that follow one behind the other behind the axis X₁₂ starting from the free edge 124 of the end 12. The tooth 121 is closest to the edge 124, while the tooth 123 is furthest from said edge.

The tooth 121 is defined between a surface 121a that is generally perpendicular to the axis X₁₂ and a surface 121b that is inclined relative to said axis by an angle β of about 40°. In practice the value of the angle β is equal to that of the angle α, and lies in the range 30° to 60°. The surface 121b comes close to a portion 126 of the end 12, from which the teeth 121 to 123 extend and that extends the portion 13, going towards the edge 124.

The teeth 122 and 123 are of substantially the same shape as the tooth 121, and each of them is likewise defined between a surface 122a or 123a that is generally perpendicular to the axis X₁₂ and a surface 122b or 123b that is inclined relative to said axis by the same angle β, and in the same direction as the surface 121b.

Between them, the teeth 121 and 122 define a volume V₁ of shape that is substantially complementary to the shape of the tooth 116. In the same manner, the teeth 122 and 123 define between them a volume V₂ of shape substantially complementary to the shape of the tooth 116.

The end 12 is also provided with a hole 125 formed between the free edge 124 and the tooth 121, and in which it is possible to insert a suture 2 represented by dashed lines solely in FIGS. 6 to 8, the suture serving to exert a traction force F on the end 12. In a variant, the suture may be replaced by other means for applying traction to the end 12, e.g. a loop constituting a silicone catheter. It is even possible to make provision for the end 12 to be molded together with an integral pull ring.

Thus, starting from the configuration of FIG. 2, it is possible to insert the edge 124 of the end 12 into the opening 115 via the side 115a of said opening that can be seen in FIG. 3, and that is opposite from the portion 13. By causing the suture 2 to pass through the opening 115, it is possible to recover the suture from the side of the opening 115 that is visible in FIG. 2 and then to pull on the end 12 by applying the force F by means of the suture 2, thereby causing the surface 121b to bear against the surface 116b. Given the direction of inclination of the surfaces 116b and 121b respectively relative to the axes X₁₁₅ and X₁₂, it is possible for sliding movement to take place between the teeth 116 and 121 by elastically deforming certain component portions of the ends 11 and 12, given that the inclination directions of the surfaces 116b and 121b are compatible with the end 12 advancing through the opening 115 under drive from the force F.

This makes it possible for the tooth 121 to pass right through the opening 15 and to bring the end 12 into a first closed configuration of the ring 1, as shown in FIG. 7. In this configuration, the mean inside diameter of the ring 1 has a first value d₁. The tooth 116 is then engaged in the volume V₁ where it prevents any withdrawal of the end 12 from the opening 115 in a direction opposite to the previously-performed movement, by virtue of its surface 116a coming to bear against the surface 121a of the tooth 121. The fact that the volume V₁ is substantially complementary in shape to the tooth 116 further improves the resulting blocking effect.

If the surgeon judges this to be necessary, continued application of the force F enables the end 12 to be moved further into the opening 115 so as to reach the position of FIGS. 3 to 6 in which the tooth 116 is engaged in the volume V₂. The inside diameter of the ring 1 then has a value d′₁ that is less than the value d₁.

If an even smaller mean diameter needs to be achieved, it is possible to continue applying the force F so as to engage the end 112 even more deeply in the opening 115 and reach the position of FIG. 8, where the tooth 116 bears within a volume V₃ adjacent to the tooth 123 opposite from the edge 124, against the surface 123a of the tooth 123. The inside diameter of the ring 1 then has a value d″₁ that is less than the value d′₁.

The mean diameter of the ring 1 is equal to the mean of the diameters of the generally circular closed collar formed by the portion 13 and the ends 11 and 12 that are mutually engaged in the closed position of the ring, it being understood that the collar is not exactly circular.

Thus, the teeth 116 and 121 to 123 that are provided respectively on the ends 11 and 12 enable the end 112 to be held stationary relative to the opening 115 in each of the three configurations shown respectively in FIGS. 6 to 8, thereby enabling the inside diameter of the ring 1 to be adjusted when in the closed configuration to its implantation conditions.

Each of the partitions 111 and 112 is pierced by a respective orifice 111c and 112c, these orifices being in alignment in a direction Y₁₁ that is perpendicular to the axis 115.

Furthermore, the end 111 is provided with three holes 121c, 122c, and 123c that are formed respectively in register with the teeth 121 to 123, in the portion 126 of the end 12 that extends the portion 13.

The holes 121c, 122c, and 123c extend in directions perpendicular to the axis X₁₂, and they are disposed relative to the teeth 121 and 122 in such a manner that the orifice 121c is aligned on the direction Y₁₁ in the configuration shown in FIG. 7, while the orifice 122c is aligned on the direction Y₁₁ in the configuration of FIG. 6, and the orifice 123c is aligned on the direction Y₁₁ in the configuration of FIG. 8.

Thus, in each of the configurations shown in FIGS. 6 to 8, one of the holes 121c, 122c, and 123c co-operates with the orifices 111c and 112c to form a channel in which it is possible to insert means for retaining or locking ends 11 and 12 in the corresponding configuration. For example, a suture 3 can be inserted in the resulting channel, as can be seen in FIGS. 1 and 9 only, prior to knotting the suture around the ends 11 and 12 that are thus held stationary and locked relative to each other.

In a variant, instead of using a suture, it is possible to use a staple or any other appropriate stop means that do not run the risk of injuring the gastric wall.

In a variant of the invention that is not shown, a single hole could be provided in the end 12, with three adjacent orifices being formed in the partitions 111 and 112, the hole in the end 12 being brought into register with one of the sets of orifices in order to form a channel for passing a holding member, depending on the selected locking position.

Under all circumstances, the hole(s) formed in the end 12 constitute(s) an orifice for passing a tie or a member for locking the ends 11 and 12 relative to each other.

Reference P₁ designates the midplane of the ring 1 that constitutes the plane on which the section of FIG. 6 is taken. The axes X₁₁₅ and X₁₂ lie in this plane. The portion 13 is provided at each of its longitudinal ends 131 and 132 and on either side of the plane P₁ with a respective lug 133, 134 having formed therein a respective orifice 135, 136 for passing a suture for ligating the ring 1 to the portion P of the stomach. These orifices 135 and 136 are substantially diametrically opposite when the ring is in the closed configuration. These orifices lie on either side of the plane P₁ such that regardless of the approach used for placing the ring 1, one of these orifices is accessible to the surgeon for the purpose of putting a ligating suture 4 into place to prevent movement relative to the portion P.

This distribution of the orifices 135 and 136 facilitates the work of the surgeon. Depending on the direction in which the ring 1 is put into place, as shown in FIGS. 1 and 9, if it is assumed that the surgeon approaches the stomach E from the direction in which these figures are viewed, then the surgeon can make use of the orifice 135 (FIG. 1) or of the orifice 136 (FIG. 9) for the purpose of ligating the ring 1 to the portion P.

It should be observed that the ring 1 may be also be implanted with its ends 11 and 12 engaged in the cutout D while having its orifice 135 accessible to the surgeon, as in the configuration of FIG. 1.

The invention is described above with reference to a ring 1 having three locking positions in which it is possible to prevent the end 12 moving relative to the end 11. Nevertheless, the invention is applicable to a ring provided with only two locking positions, or on the contrary to a ring provided with more than three locking positions. The number of teeth on the end 12 is adapted to the number of locking positions it is desired to obtain.

The invention is shown for a ring that is used in a calibrated vertical gastroplasty operation. Nevertheless, the invention is applicable to a ring for use in calibrating a portion of the stomach prior to anastomosis, for the purpose of providing a gastric short circuit, or for a ring that is used for calibrating the pylorus of a patient.

1. A gastric ring (1) having no expandable portion, said ring having two ends (11, 12) and, between said ends, an intermediate portion (13) of solid section for bearing simply against a portion (P) of a stomach (E) to be surrounded, a first end (11) forming an opening (115) in which it is possible to insert the second end (12), said ring including portions in relief (116, V₁-V₃) for blocking said second end (12) in said opening (115) in at least two distinct positions (FIGS. 6 to 8), the ring being characterized in that it comprises, in addition to said portions in relief (116, V₁, V₂, V₃), means (111c, 112c, 121c, 122c, 123c, 3) for locking said second end (12) in said opening (115) in each of said positions, said locking means comprising at least one orifice (111c, 112c) formed in one (11) of said ends, and at least two second orifices (121c, 122c, 123c) formed in the other end (12), one of said second orifices being in alignment with said first orifice in each of said positions, said orifices then together forming a channel for passing a holding member (3) for holding said ends relative to each other.

2. A ring according to claim 1, characterized in that it includes two first orifices (111c, 112c) formed in two partitions (111, 112) belonging to the first end (11) and between which said opening (115) is defined, and in that said two second orifices (111c, 112c) are in alignment on a direction (Y₁₁) perpendicular to a longitudinal axis (X₁₁₅) of said opening.

3. A ring according to claim 1, characterized in that said opening (115) is provided with a blocking portion in relief (116) suitable for co-operating, as a function of the diameter selected for said ring (1), with one of a plurality of complementary blocking portions in relief (V₁, V₂, V₃) formed on said second end (12).

4. A ring according to claim 3, characterized in that said blocking portion in relief of said opening is a tooth (116) projecting from an edge (114) of said opening (115) towards the opposite edge (113).

5. A ring according to claim 3, characterized in that said complementary portions in relief (V₁, V₂, V₃) formed on said second end (12) are formed by teeth (121, 122, 123) disposed one behind another along a longitudinal axis (X₁₂) of said second end.

6. A ring according to claim 5, characterized in that said second orifices (121c, 122c, 123c) are formed respectively at the level of said teeth (121, 122, 123) of said second end (12), in a portion (126) of said second end (12) that extends said intermediate portion (13), and in that said orifices extend in directions that are perpendicular to a longitudinal axis (X₁₂) of said second end.

7. A ring according to claim 5, characterized in that said teeth (116, 121, 122, 123) have inclined surfaces (116b, 121b, 122b, 123b) that are inclined, respectively relative to a middle axis (X₁₁₅) of said opening (115) and relative to the longitudinal axis (X₁₂) of said second end (12), in directions that are compatible with said second end moving through said opening in a direction compatible with tightening the ring.

8. A ring according to claim 7, characterized in that said teeth (116, 121, 122, 123) have respective surfaces (116a, 121a, 122a, 123a) that are perpendicular to said middle axis (X₁₁₅) of said opening (115) and to said longitudinal axis (X₁₂) of said second end (12), said surfaces bearing one against another when said second end is prevented from moving in said opening in one of said positions.

9. A ring according to claim 4, characterized in that a volume (V₁, V₂) defined between two adjacent teeth (121, 122, 123) formed on said second end (12) is substantially complementary to the tooth (116) that projects from an edge (114) of said opening (115).

10. A ring according to claim 1, characterized in that said intermediate portion (13) is provided, on each of its longitudinal edges (131, 132), with an orifice (135, 136) for passing a ligating suture (4), said orifices being substantially diametrically opposite when said second end (12) is prevented from moving in said opening (115) in one of said positions.
